(19) United States
(12) Patent Application Publication
(10) Pub. No.: US 2009/0198247 A1
(43) Pub. Date: Aug. 6, 2009

(54) INTRAOCULAR LENS IMPLANTATION KIT

(75) Inventor: Joshua Ben Nun, Vitkin (IL)

Correspondence Address: MORGAN LEWIS & BOCKIUS LLP, 1111 PENNSYLVANIA AVENUE NW, WASHINGTON, DC, 20004, US

(73) Assignee: NuLens Ltd.

(21) Appl. No.: 12/438,830

(22) PCT Filed: Aug. 26, 2007
(86) PCT No.: PCT/IL2007/001056

§ 371 Date: Feb. 25, 2009

Related U.S. Application Data
(60) Provisional application No. 60/823,580, filed on Aug. 25, 2006.

(51) Int. Cl. A61F 9/007; A61B 17/00

(52) U.S. Cl. 606/107

(57) ABSTRACT

Intraocular lens (IOL) implantation kit for assisting in implanting an accommodating intraocular lens (AIOL) assembly in a human eye. The AIOL assembly includes an AIOL having a rigid tubular main body with opposite leading and trailing surfaces, and a haptics system having a leading haptics and a trailing haptics for forced insertion into a pair of generally diametrically opposite segments in a human eye's ciliary sulcus. The IOL implantation kit includes a forceps-like insertor tool for clamping an AIOL heightwise between its leading and trailing surfaces for assisting in anchoring its leading haptics in a distal segment of a human eye's ciliary sulcus. The implantation kit includes a crook-like insertor tool for assisting in fixating an AIOL assembly's trailing haptics generally diametrically opposite its leading haptics.

### FIELD OF THE INVENTION

The invention pertains to intraocular lens implantation kits.

### BACKGROUND OF THE INVENTION

Commonly owned PCT International Application No. PCT/IL2005/000456 entitled Accommodating Intraocular Lens Assemblies and Accommodation Measurement Implant and published under PCT International Publication No. WO 2005/104994 illustrates and describes accommodating intraocular lens (hereinafter “AIOL”) assemblies, the contents of which are incorporated by reference. The AIOL assemblies include a haptics system adapted to be securely fixated in a human eye's ciliary sulcus at least two spaced apart stationary anchor points so that it may act as a reference plane for an AIOL of continuously variable strength affected by a human eye's capsular diaphragm acting thereagainst from a posterior direction and under the control of its sphincter-like ciliary body. The haptics system preferably includes self-anchoring haptics as illustrated and described in commonly owned PCT International Application No. PCT/IL02/00128 entitled Intraocular Lens and published under PCT International Publication No. WO 02/065951. The haptics terminate at attachment plates preferably including positioning holes for facilitating fixation in a human eye's ciliary sulcus (see W0 02/065951's FIG. 1). The positioning holes are preferably throughgoing.

### SUMMARY OF THE INVENTION

The present invention is directed towards an intraocular lens (hereinafter “IOL”) implantation kit for assisting in the implantation of AIOL assemblies in human eyes. The IOL implantation kit includes a forceps-like insertor tool manually operative between an unclamping state and a manually urged clamping state for securely clamping an AIOL heightwise for securely holding an AIOL assembly for assisting in anchoring its leading haptics in a distal segment of a human eye's ciliary sulcus. The IOL implantation kit also includes a crook-like insertor tool including a handheld shank having a leading end with a U-shaped hook delimiting an open ended elongated slot having an open end towards its handheld shank for fitting around a human eye's iris. The U-shaped hook includes a hook tip extending downward from the U-shaped slot's open end for removable insertion into a trailing haptics' positioning hole for assisting in anchoring the trailing haptics in a proximal segment of a human eye's ciliary sulcus. The crook-like insertor tool can be provided with either a clockwise or counter-clockwise angled U-shaped hook in a top view of the tool. The insertor tools are formed from bio-compatible medical grade durable rigid materials in general, and stainless steel in particular. The insertor tools can be prepared from heat or irradiation stable materials for re-use or prepared as disposable items for single use applications.

### BRIEF DESCRIPTION OF THE DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a cross section view of an anterior part of a human eye in its contracted ciliary body state for natural near vision in an axial plane of the human body;

FIG. 2 is a cross section view of an anterior part of a human eye in its relaxed ciliary body state for natural distance vision in an axial plane of the human body;

FIG. 3 is a pictorial view of an AIOL assembly with a pair of oppositely extending C-shaped haptics for self-anchoring in a human eye's ciliary sulcus;

FIG. 4 is a top view of FIG. 3's AIOL assembly;

FIG. 5 is a pictorial view of a forceps-like insertor tool in an unclamping state;

FIG. 6 is a pictorial view of FIG. 5's insertor tool in a manually urged clamping state;

FIG. 7 is a side view of FIG. 5's insertor tool in its unclamping state;

FIG. 8 is a side view of FIG. 5's insertor tool in its manually urged clamping state;

FIG. 9 is a close-up pictorial view of FIG. 5's insertor tool's lower clamping arm's free end showing its tip with its generally L-shaped construction;

FIG. 10 is a close-up pictorial view of FIG. 5's insertor tool's upper clamping arm's free end showing its tip with its generally inverted L-shaped construction;

FIG. 11 is a pictorial view showing FIG. 5's insertor tool securely clamping FIG. 3's AIOL heightwise for securely holding the AIOL assembly;

FIG. 12 is a pictorial view of a crook-like insertor tool with a U-shaped hook;

FIG. 13 is a side view of FIG. 12's insertor tool;

FIG. 14 is a top view of FIG. 12's insertor tool with a clockwise angled U-shaped hook;

FIG. 15 is a top view of FIG. 12's insertor tool with a counter clockwise angled U-shaped hook;

FIG. 16 is a close-up view of FIG. 12's insertor tool's U-shaped hook;

FIG. 17 is a pictorial view of FIG. 12's insertor tool engaging FIG. 3's AIOL assembly's trailing haptics;

FIG. 18 is a close-up pictorial view of FIG. 12's insertor tool's U-shaped hook engaging FIG. 3's AIOL assembly's trailing haptics' positioning hole;

FIG. 19 is a vertical cross section of the anterior segment of a human eye showing FIG. 12's insertor tool holding FIG. 3's SOL assembly for inserting the leading haptics under the human eye's iris into its ciliary sulcus;

FIG. 20 is the same vertical cross section showing the insertion of FIG. 12's insertor tool's hook tip into FIG. 3's AIOL assembly's trailing haptics' positioning hole as the trailing haptics rests on the edge of a corneal incision made in the human eye;

FIG. 21 is the same vertical cross section showing the use of FIG. 12's insertor tool for fixating FIG. 3's AIOL assembly's trailing haptics in a proximate segment of the human eye's ciliary sulcus;

FIG. 22 is the same vertical cross section showing FIG. 3's AIOL assembly anchored in the human eye; and

FIG. 23 is a three dimensional view of FIG. 3's AIOL assembly anchored in the human eye.

### DETAILED DESCRIPTION OF PREFERRED EMBODIMENTS OF THE PRESENT INVENTION

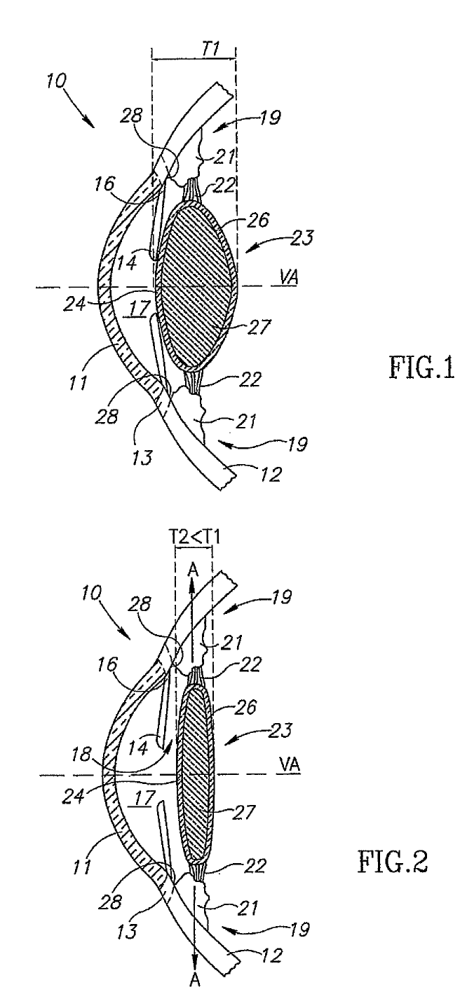

FIGS. 1 and 2 are cross section views of an anterior part of a human eye 10 having a visual axis VA in its natural near and distance vision conditions, respectively, in an axial plane of the human body. The human eye 10 has a cornea 11 peripherally connected to a spherical exterior body made of tough connective tissue known as the sclera 12 at an annular sclero-corneal juncture 13. An iris 14 inwardly extends into the human eye 10 from its root 16 at the sclero-corneal juncture 13 to divide the human eye's anterior part into an anterior chamber 17 and a posterior chamber 18. A sphincter-like peripheral structure known as the ciliary body 19 includes ciliary processes housing ciliary muscles 21 fired by parasympathetic nerves. The ciliary muscles 21 are connected to zonular fibers 22 which in turn are peripherally connected to the equatorial edge of a membrane known as the capsular bag 23 with an anterior capsule 24 and a posterior capsule 26 enrobing a natural crystalline lens 27. The iris's root 16 and the ciliary body 19 delimit a portion of the interior surface of the sclera 12 at the sclero-corneal juncture 13 known as the ciliary sulcus 28. Remnants of the anterior capsule 24 which may remain after extraction of the natural crystalline lens 27 and the intact posterior capsule 26 are referred to hereinafter as the capsular diaphragm 29. Contraction of the ciliary body 19 allows the lens 27 to thicken to its natural thickness T1 along the visual axis VA for greater positive optical power for near vision (see FIG. 1). Relaxation of the ciliary body 19 tensions the zonular fibers 22 which draws the capsular bag 23 radially outward as shown by arrows A for compressing the lens 27 to shorten its thickness along the visual axis VA to T2<T1 for lower positive optical power for distance vision (see FIG. 2).

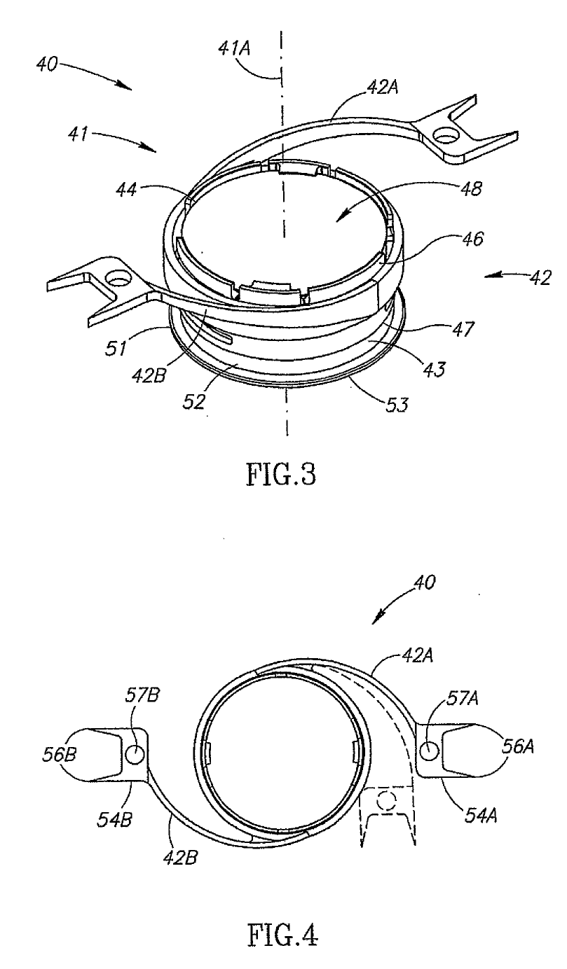

FIGS. 3 and 4 show an AIOL assembly 40 with an AIOL 41 and a haptics system 42 including a pair of oppositely extending C-shaped haptics 42 including a leading haptics 42A and a trailing haptics 42B for self-anchoring in a human eye's ciliary sulcus. The AIOL 41 has a longitudinal axis 41A and includes a rigid tubular main body 43 with an annular leading surface 44, a peripheral surface 46, and an annular tailing surface 47 generally parallel and opposite to the leading surface 44. The AIOL 41 has a leading rigid optical element 48, a shape memory optical element 49 (not shown) housed in the main body 43, and a piston member 51 with a peripheral surface 52 and a trailing surface 53 spaced apart from the main body's trailing surface 47 and reciprocal with respect thereto for changing the optical element 49's Diopter strength. The main body's leading surface 44 and the piston member's trailing surface 53 correspondingly face in an anterior direction and a posterior direction on implanting the AIOL assembly 40 in a human eye 10. The AIOL 41 has a diameter D≈6±0.5 mm. The AIOL 41 has a height H≈3±0.5 mm between its leading surface 44 and trailing surface 47.

The haptics 42A and 42B terminate in attachment plates 54A and 54B each respectively provided with a pair of rigid puncturing members 56A and 56B and a positioning hole 57A and 573. Positioning holes 57A and 57B are preferably throughgoing holes. The haptics 42A and 42B are rigid along the longitudinal axis 41A and flexible therearound such that they can encircle around the main body 43 as shown in dashed lines for facilitating insertion of the AIOL assembly 40 through a corneal incision into a human eye 10. The puncturing members 56A and 56B are designed to puncture the tough connective tissue of a human eye's scleral wall for anchoring purposes.

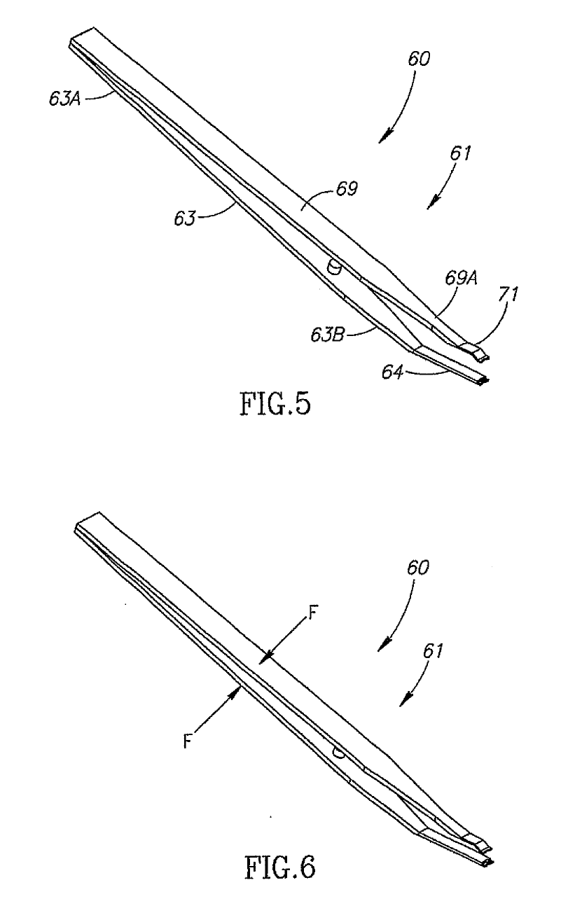

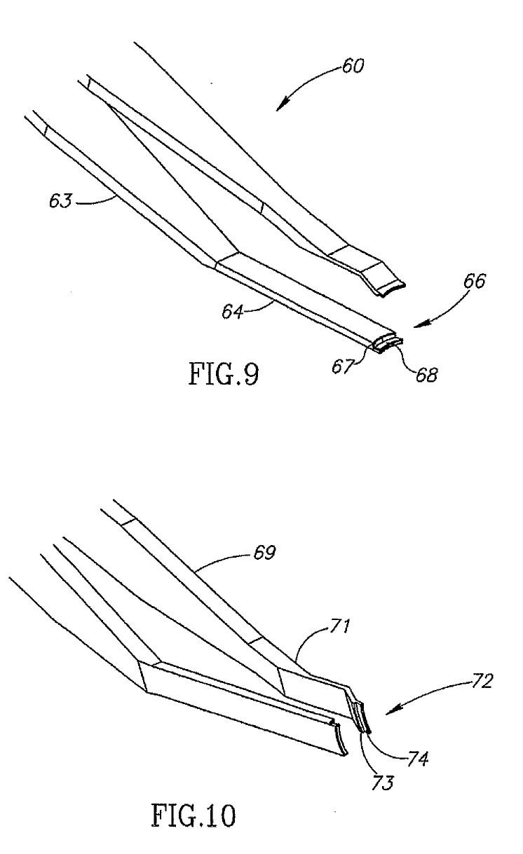

FIGS. 5 to 11 show a forceps-like insertor tool 60 for securely clamping the AIOL assembly 40 and assisting in fixating its leading haptics 42A in a distal segment of a human eye's ciliary sulcus 28. The forceps-like insertor tool 60 includes a handheld elongated body member 61 having a longitudinal axis 62 and designed to be manipulated on application of a clamping force denoted F from an unclamping state to a manually urged clamping state for securely clamping the AIOL 41 heightwise between its leading surface 44 and its trailing surface 47 for securely holding the AIOL assembly 40.

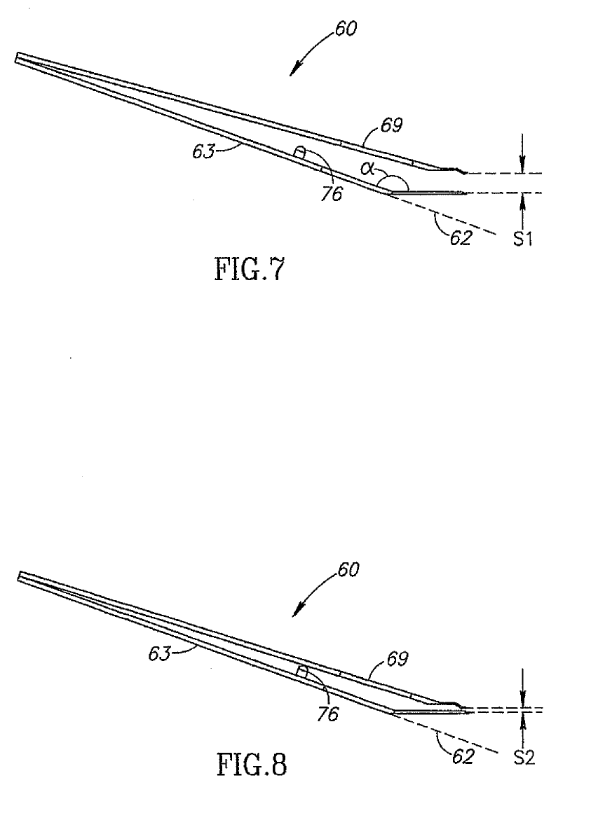

The elongated body member 61 includes a lower clamping arm 63 defining the longitudinal axis 62 and having a trailing end 63A and a leading end 63B. The leading end 63B has a free end 64 inclined with respect to the lower clamping arm 63 to subtend an obtuse included angle α≈160±5° (see FIG. 7). The lower clamping arm 63 has a length L≈80 mm and the free end 64 has a length L≈16 mm. The free end 64 terminates in a tip 66 with a generally L-shaped construction including a leading surface 67 and a lower clamping surface 68 for respectively bearing against the AIOL's main body's peripheral surface 46 and its annular trailing surface 47 (see FIG. 11).

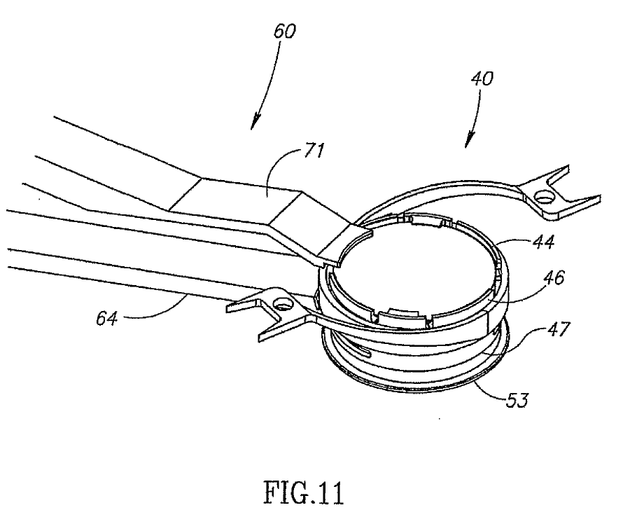

The elongated body member 61 includes an upper clamping arm 69 flexibly mounted on the lower clamping member 63. The upper clamping arm 69 has a leading end 69A with a free end 71 distanced from and generally parallel to the lower clamping arm's free end 64. The free end 71 terminates in a tip 72 with a generally inverted L-shaped construction including a leading surface 73 and an upper clamping surface 74 for respectively bearing against the AIOL's main body's peripheral surface 46 and its leading surface 44 (see FIG. 10).

The lower clamping arm 63 is formed with a stopper 76 towards its free end 64 for stopping the upper clamping arm's free end 71 directly bearing on the former's free end 64 in the manually urged clamping state to prevent damage to same. The free ends 64 and 71 have a separation S1≈5.3±0.5 mm designed to be greater than the AIOL's height in the insertor tool's unclamping state for conveniently enabling the AIOL 41 to be inserted therebetween (see FIG. 7). The free ends 64 and 71 have a separation S2≈0.5±0.1 mm designed to be less than the AIOL's height in the insertor tool's manually urged clamping state (see FIG. 8).

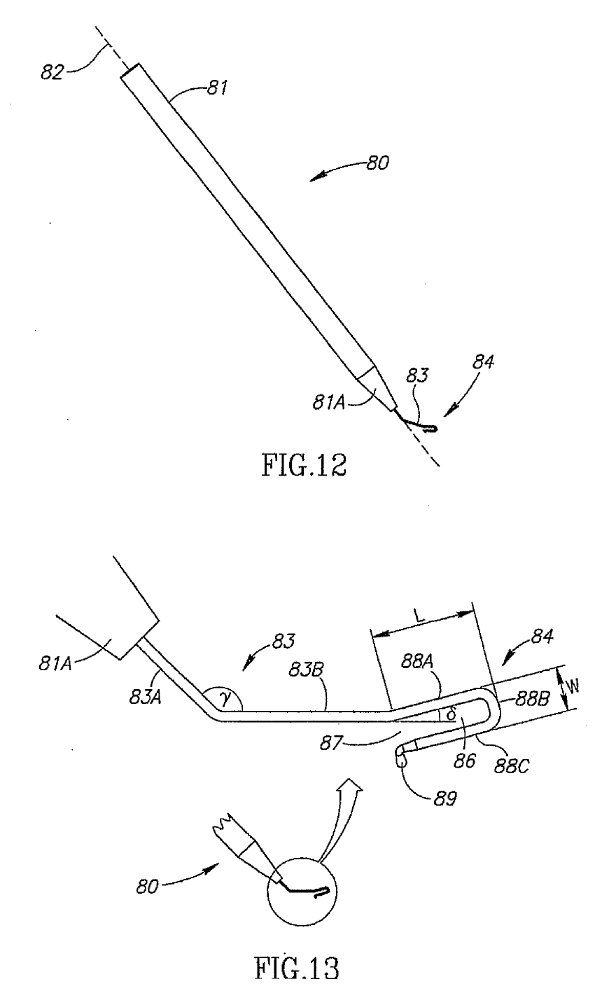

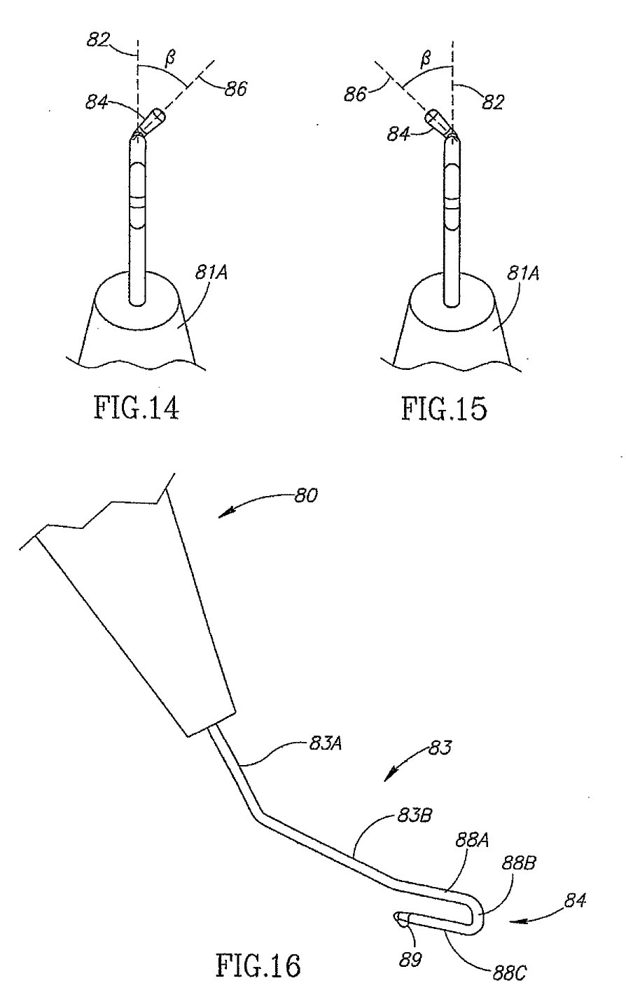

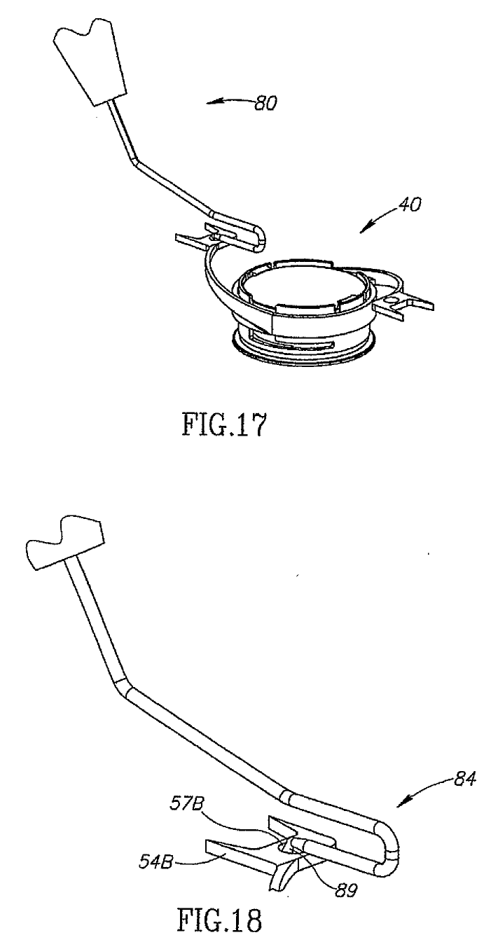

FIGS. 12 to 18 show a crook-like insertor tool 80 for assisting in fixating the AIOL's trailing haptics 42B in a proximal segment of the human eye's ciliary sulcus 28. The crook-like insertor tool 80 includes a handheld shank 81 with a longitudinal axis 82. The handheld shank 81 has a leading end 81A having an extension 83 terminating in a U-shaped hook 84. The U-shaped hook 84 defines a longitudinal axis 86 inclined with respect to the longitudinal axis 82 at an acute angle β≈45±5° in a top view of the crook-like insertor tool 80. The crook-like insertor tool 80 can be provided with either a clockwise or counter-clockwise angled U-shaped hook 84 as shown respectively in FIGS. 14 and 15.

The extension 83 includes a first extension segment 83A adjacent the handheld shank's leading end 81A and a second extension segment 83B adjacent the U-shaped hook 84. The extension segment 83A is generally co-directional with the handheld shank's longitudinal axis 82 in a side view of the crook-like insertor tool 80 (see FIG. 13). The extension segment 83B is inclined with respect to the extension segment 83A at an obtuse included angle γ≈135±10° in the insertor tool's side view (see FIG. 13).

The U-shaped hook 84 delimits an open ended elongated slot 86 having an open end 87 facing toward the handheld shank 81 for fitting around a human eye's iris. The U-shaped hook 84 has a first leg 88A connected to the extension segment 83B, a second leg 88B generally perpendicular to the first leg 88A and a third leg 88C generally parallel and opposite the first leg 88A. The slot 86 has a slot length L≈3±0.5 mm and a slot width W≈1±0.3 mm. The U-shaped hook 84 includes a hook tip 89 extending downward from its open end 87 in the insertor tool's side view (see FIG. 13) for removable insertion into a trailing haptics' positioning hole 57B for assisting in anchoring same in a proximal segment of a human eye's ciliary sulcus. The U-shaped hook's longitudinal axis 86 is inclined with respect to the extension segment 83B at an obtuse included angle δ≈15±5°.

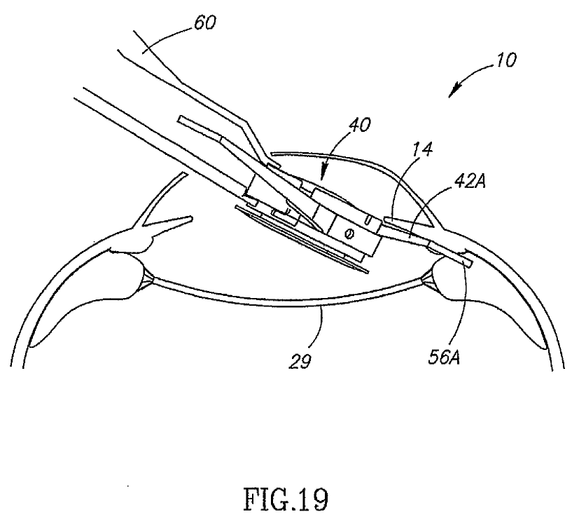

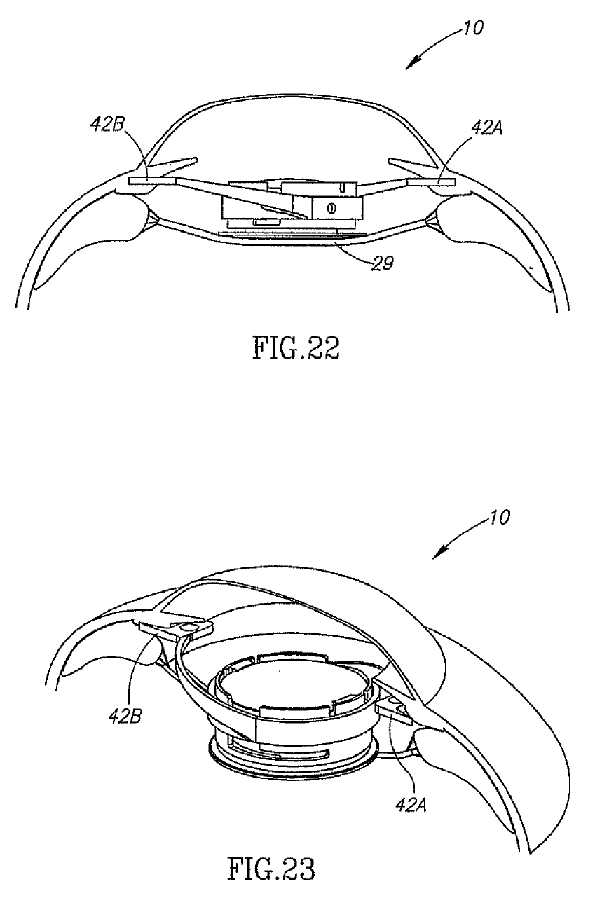

FIGS. 19 to 23 show the use of the IOL implantation kit of the present invention for assisting in the implantation of the AIOL assembly 40 in a human eye 10 after removal of its lens content and filling its anterior segment with viscoelastic material.

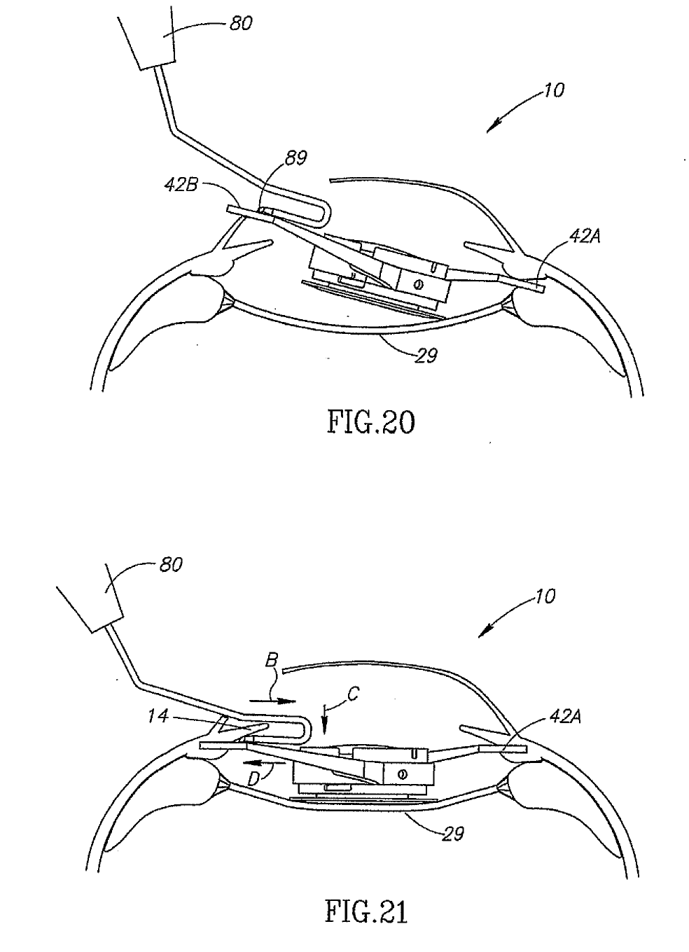

FIG. 19 shows the forceps-like insertor tool 60 holding the AIOL assembly 40 for inserting the leading haptics 42A under the iris 14 into the ciliary sulcus 28. The surgeon pushes the forceps-like insertor tool 60 forwards such that the AIOL assembly's leading haptics' attachment plate's puncturing members 56A overcome the natural resistance of the connective tissue thereby anchoring the leading haptics 42A therein. Once the leading haptics 42A is positioned in the ciliary sulcus 28, the surgeon rests the AIOL 41 on the collapsed capsular bag 23 and its trailing haptics 42B on the edge of the corneal incision and detaches the forceps-like insertor tool 60 from the AIOL assembly 40. In this position, the surgeon inserts the crook-like insertor tool's hook tip 89 into the trailing haptics' attachment plate's positioning hole 57B (see FIG. 20).

FIG. 21 shows the use of the crook-like insertor tool 80 for fixating the AIOL assembly's trailing haptics 42B in a proximate segment of the human eye's ciliary sulcus. The surgeon initially gently urges the crook-like insertor tool 80 towards the distal segment in which the leading haptics 42A is anchored denoted by arrow B. Once the surgeon reaches the edge of the iris, the surgeon gently urges the U-shaped hook 84 downwards towards the capsular diaphragm 29 until the trailing haptics' attachment plate 54B3 is below the iris 14 at the proximal side denoted by arrow C. The AIOL 41 bears against the capsular diaphragm 29 which conforms to its shape thereby tensioning same. The surgeon pulls the crook-like insertor tool 80 towards himself below the iris' proximal segment (as denoted by arrow D), thereby pushing the trailing haptics 42B towards the ciliary sulcus' proximal segment while the iris' proximal segment extends into the U-shaped hook 84. Once the surgeon anchors the trailing haptics 42B in the ciliary sulcus' proximal segment, the surgeon releases the hook tip 89 from the positioning hole 57B by a gentle sideward tilt and ten moves the crook-like insertor tool 80 towards the center of the anterior chamber 17 above the iris 14 and outside the eye 10. The viscoelastic is flushed from the anterior chamber 17 and the incision is sutured.

While the invention has been described with respect to a limited number of embodiments, it will be appreciated that many variations, modifications, and other applications of the invention can be made within the scope of the appended claims.

1. A forceps-like insertor tool for use in implanting an accommodating intraocular lens (AIOL) assembly into a human eye having a ciliary sulcus,
the AIOL assembly including (a) an AIOL having a rigid tubular main body with an annular leading surface, a peripheral surface and an annular trailing surface generally opposite and parallel to the annular leading surface, and (b) a haptics system having a pair of oppositely directed haptics each terminating in an attachment plate with at least one puncturing member for forced insertion into the human eye's ciliary sulcus,
the forceps-like insertor tool comprising a handheld elongated body member having a longitudinal axis and manually operative between an unclamping state and a manually urged clamping state for securely clamping the AIOL heightwise between its leading and trailing surfaces for securely holding the AIOL assembly for assisting in anchoring its leading haptics in a distal segment of a human eye's ciliary sulcus on pushing the tool forward for causing the leading haptics' attachment plate to be anchored in the ciliary sulcus,
said handheld elongated body member including
(i) a lower clamping arm defining said longitudinal axis and having a trailing end and a leading end, said lower clamping arm's leading end having a free end subtending an obtuse included angle α with respect to said longitudinal axis in a side view of the forceps-like insertor tool and terminating in a tip with a generally L-shaped construction including a leading surface and a lower clamping surface for respectively bearing against the rigid tubular main body's peripheral surface and trailing surface in said manually urged clamping state, and
(ii) an upper clamping arm flexibly mounted on said lower clamping arm and having a leading end with a free end distanced from and generally parallel to said lower clamping arm's free end in said unclamping state, said upper clamping arm's free end terminating in a tip with a generally inverted L-shaped construction including a leading surface and an upper clamping surface for respectively bearing against the rigid tubular main body's peripheral surface and leading surface in said manually urged clamping state
wherein said lower clamping arm's lower clamping surface and said upper clamping arm's upper clamping surface having a separation respectively greater than and less than the AIOL's height in said unclamping state and said manually urged clamping state.

2. The tool according to claim 1 wherein said handheld elongated body member includes a stopper between said lower clamping arm and said upper clamping arm for stopping said upper clamping arm's free end directly bearing on said lower clamping arm's free end in said manually urged clamping state.

3. The tool according to either claim 1 or 2 wherein said obtuse included angle α≈160±5°.

4. A crook-like insertor tool for use in implanting an accommodating intraocular lens (AIOL) assembly into a human eye having a ciliary sulcus and an iris,
the AIOL assembly including an AIOL and a haptics system having a pair of oppositely directed haptics each terminating in an attachment plate with at least one puncturing member for forced insertion into the human eye's ciliary sulcus, a trailing haptics of said pair of oppositely directed haptics having a positioning hole for enabling manipulation of its attachment plate with respect to the human eye,
the crook-like insertor tool comprising a handheld shank defining a longitudinal axis and having a leading end with a U-shaped hook delimiting an open ended elongated slot having an open end facing toward said handheld shank, and shaped and dimensioned for fitting around the human eye's iris,
said U-shaped hook including a hook tip extending downward from said open end in a side view of the crook-like insertor tool for removable insertion into the trailing haptics' positioning hole for assisting in anchoring the trailing haptics in a proximal segment of a human eye's ciliary sulcus.

5. The tool according to claim 4 wherein said elongated slot had a slot length L≈3±0.5 mm and a slot width W≈1±0.3 mm.

6. The tool according to either claim 4 or 5 wherein said U-shaped hook defines a longitudinal axis subtending an acute angle β≈45±5° with respect to said handheld shank's longitudinal axis in a top view of the crook-like insertor tool.

7. The tool according to any one of claims 4 to 6 and further comprising an extension intermediate said handheld shank and said U-shaped hook, said extension including a first extension segment adjacent said handheld shank and a second extension segment adjacent said U-shaped hook, said first extension segment being generally co-directional with said handheld shank's longitudinal axis and said second extension segment inclined with respect to said first extension segment at an obtuse included angle γ≈135±5° in a side view of the crook-like insertor tool.

8. The tool according to claim 7 wherein said U-shaped hook's longitudinal axis subtends an obtuse included angle δ≈15±5° with respect to said second extension segment in a side view of the crook-like insertor tool.

9. A method for implanting an accommodating intraocular lens (AIOL) assembly into a human eye having a ciliary sulcus and an iris,
the AIOL assembly including an AIOL having (a) a rigid tubular main body with an annular leading surface, a peripheral surface and an annular trailing surface generally opposite and parallel to the annular leading surface, and (b) a haptics system having a pair of oppositely directed haptics each terminating in an attachment plate with at least one puncturing member for forced insertion into the human eye's ciliary sulcus, a trailing haptics of the pair of oppositely directed haptics having a positioning hole for enabling manipulation of its attachment plate with respect to the human eye,
(a) providing a forceps-like insertor tool according to any one of claims 1 to 3 for securely holding the AIOL assembly for assisting in anchoring its leading haptics in a distal segment of a human eye's ciliary sulcus on pushing the tool forward for causing the leading haptics' attachment plate to be anchored in the ciliary sulcus; and
(b) providing a crook-like insertor tool according to any one of claims 4 to 8 for assisting in anchoring the trailing haptics in a proximal segment of a human eye's ciliary sulcus generally diametrically opposite the distal segment.
